(19) United States
(12) Patent Application Publication
(10) Pub. No.: US 2020/0246127 A1
(43) Pub. Date: Aug. 6, 2020

(54) BIOLOGICALLY MODIFIED VASCULAR GRAFTS FOR IMPROVED BYPASS SURGERY OUTCOMES

(71) Applicants: Lumen Therapeutics, LLC, Menlo Park, CA (US); The Board of Trustees of the Leland Stanford Junior University, Stanford, CA (US)

(72) Inventors: Paul Leo McGrane, Sioux Falls, SD (US); C. Garrison Fathman, Portonal Valley, CA (US); Jonathan Rothbard, San Francisco, CA (US)

(21) Appl. No.: 16/784,181

(22) Filed: Feb. 6, 2020

Related U.S. Application Data
(60) Provisional application No. 62/802,096, filed on Feb. 6, 2019.

(51) Int. Cl. A61F 2/07; A61L 27/18

(52) U.S. Cl. CPC A61F 2/07; C08L 77/00; A61L 2430/36; A61L 27/18

(57) ABSTRACT

The present disclosure provides a method for creating a Biologically Modified Vein Graft with improved survival by pretreating the vein to be used as a vascular graft with compositions comprising oligo-L-arginine, or salts thereof, and an organic acid or a salt thereof. The disclosure further provides methods of improving vascular vein graft survival comprising pretreating the graft for a set period of time with a set concentration of oligo-L-arginines in a buffer comprising either the organic acid or the salt thereof and flushing the BMVG before implantation with the same buffer absent the arginine oligomer. This treatment may prevent vein graft disease in the transplanted vessel.

### CROSS-REFERENCE

This application claims the benefit of U.S. Provisional Application No. 62/802,096, filed Feb. 6, 2019, which application is incorporated herein by reference.

### BACKGROUND OF THE INVENTION

Vascular bypass surgery, including coronary arterial bypass graft (CABG) and peripheral arterial bypass graft (PABG) surgery, is a common surgical procedure that involves rerouting blood flow by interposition grafting of a blood vessel around an occluded artery. For bypass surgery, a section of vein can be used as a graft and attached proximally and distally relative to the affected artery to bypass the area of occlusion.

While bypass surgery can be a very effective procedure, the vein grafts often fail due to processes that lead to re-occlusion of the transplanted graft, collectively called vein graft disease. These processes include thrombosis, intimal hyperplasia, and atherosclerosis. Although these processes may be temporally distinct, they can be pathophysiologically interconnected in the development of vein graft disease.

Because the great majority of interposition grafts that fail due to vein graft disease do so within several years of implantation, there is a need for treatment to prevent vein graft disease to improve graft survival and to reduce morbidity from failure of interposition grafts. An effective treatment to prevent vein graft disease as described herein can be the creation of a biologically modified vein graft (BMVG), prior to implantation, via the ex vivo treatment of the vein segment with oligo-L-arginine. Prior treatments utilizing oligo-L-arginine for creation of BMVGs employed phosphate buffered saline (PBS) as a vehicle. Solutions of oligo-L-arginine in PBS were shown to be safe and effective in several animal models (rat, rabbit, and porcine). Surprisingly, in a human study a solution of oligo-L-arginine in PBS was found to be inferior to historical controls, although it was superior to PBS control. Subsequent to this human study, results from ex vivo and clinical studies were published demonstrating that saline solutions, such as PBS, are not suitable vehicles for incubation of venous segments to be used as bypass conduits, consistent with the results observed for oligo-L-arginine in PBS. There was thus a need to find a solution which would be compatible with both the venous segments and the oligo-L-arginine.

### SUMMARY OF THE INVENTION

Disclosed herein are methods to improve vascular graft survival by reducing, or preventing, thrombosis, intimal hyperplasia, and atherosclerosis that lead to vein graft disease, and thereby preventing re-occlusion and failure of the graft.

Also disclosed are compositions that can be applied to the vein graft, ex vivo, prior to grafting, to create a biologically modified vein graft (BMVG) with increased nitric oxide production. An example of such a composition is a solution comprising an oligo-L-arginine in a “non-saline” vehicle. Such compositions include, e.g., a solution containing oligomers of L-arginine, or salts thereof, and an organic acid, or a salt thereof. The organic acid or the salt thereof can be a C3-C10 organic acid or salt thereof. The C3-C10 organic acid or salt thereof can be a lactic acid or a salt thereof. For example, the composition may be a solution of Lactated Ringer's Solution containing oligomers of L-arginine. The compositions may comprise at least one of a lactate ion, a sodium ion, a potassium ion, a calcium ion, and a chloride ion. The composition can consist essentially of sodium lactate, sodium chloride, potassium chloride, and calcium chloride. The compositions may be devoid of phosphate ions. The compositions may be devoid of disodium phosphate and/or a monopotassium phosphate. The pH of the compositions can be between 6.0 and 7.5. Preferably, the pH of the composition is 6.5.

In some instances, the arginine residues in the compositions herein are L-arginine residues. Such L-arginine residues may be present in an oligomer. An oligo-L-arginine is an oligomer comprising a plurality of arginine residues. In any of the embodiments herein, a composition can include a plurality of different oligo-L-arginines. In instances when the composition is a solution, concentration of the oligo-L-arginine(s) in the solution may be between 10 μM and 200 μM or 50 μM and 150 μM. In a preferred instance, the concentration of a nona-L-arginine in a solution is between 10 μM and 200 μM. Alternatively, the concentration of a nona-L-arginine in a solution is between 50 μM and 150 μM. Alternatively, the concentration of a nona-L-arginine in a solution is about 100 μM. Other embodiments include concentration of oligo-L-arginine(s) of at least 1, 5 or 10 and/or up to 200, 250, 300 or 500 μM.

The oligo-L-arginine can comprise 6 to 15 L-arginine residues, either as a homopolymer or a heteropolymer. An oligomer herein can be a nona-oligomer consisting of nine L-arginine monomers. In any of the embodiments herein, the oligo-L-arginine solution can further comprise at least one of an acetate and a trifluoroacetate. In preferred embodiments, the solution does not contain heparin. In some cases, the solution may not contain an antibiotic.

Also disclosed herein is a kit comprising a dry formulation comprising an oligo-L-arginine, a solution, and an instruction comprising written directions giving one or more steps for combining the dry formulation and the solution to form a final solution. The solution can comprise an organic acid or a salt thereof. The final solution can be any of the compositions described herein.

The methods herein involve applying any of the compositions described to a vein graft ex vivo prior to grafting. In some instances, the methods comprise contacting an ex vivo blood vessel to be grafted with a solution comprising oligo-L-arginine, or a salt thereof, and an organic acid, or a salt thereof. In some cases, the vein graft may be a human vein graft. The organic acid or the salt thereof can be a C3-C10 organic acid, or a salt thereof. The C3-C10 organic acid or a salt thereof can be lactic acid or a salt thereof. The solution can comprise at least one of a lactate ion, a sodium ion, a potassium ion, a calcium ion, and a chloride ion. The solution can consist essentially of sodium lactate, sodium chloride, potassium chloride, and calcium chloride. The solution may be devoid of a phosphate ion. The solution may be devoid of a disodium phosphate and/or a monopotassium phosphate. The pH of the solution can be between 6.0 and 7.5. Preferably, the pH of the solution is 6.5.

Solutions of oligo-L-arginine can have different concentrations of the oligo-L-arginine(s) therein. In some instances, the concentration of the oligo-L-arginine in the solution can be between 50 μM to 150 μM. Alternatively, the oligo-L-arginine concentration is about 100 μM. In such instances, the oligo-L-arginine is a nona-L-arginine. The oligomers of arginine may comprise from 6- to 15 L-arginine residues. The oligo-L-arginine solution can further comprise an acetate. The solution can be devoid of heparin. The solution may further be devoid of an antibiotic.

Any of the solutions herein can be contacted with a graft for a set amount of time. The contacting can occur for at least 5 minutes and up to about 60, 55, 50, 45, 40, 35, 30, 25, 20, 15, or 10 minutes. In some instances, the contacting of the vein graft and the solution occurs for up to about 45 minutes. Alternatively, the contacting of the vein graft and the solution occurs for up to about 15 minutes. The preferred contact time is about 10 minutes.

Contacting the blood vessel with the solution can result in inhibition of thrombosis, intimal hyperplasia, and/or atherosclerosis. The contact can result in increased nitric oxide production. Hence, a method of increasing nitric oxide production in a vascular graft ex vivo can comprise contacting the vascular graft, prior to grafting, with a solution comprising an oligo-L-arginine, or salt thereof, and an organic acid, a salt thereof. The organic acid or the salt thereof can be a C3-C10 organic acid or the salt thereof. The C3-C10 organic acid or the salt thereof can be lactic acid or a salt thereof.

The blood vessel to be grafted can be a saphenous vein, for example, which can be taken from the lower leg. In any of the methods herein, the vein graft can be used as a coronary arterial bypass graft (CABG). Alternatively, the vascular graft can be used as a peripheral arterial bypass graft (PABG).

The methods disclosed involve the steps of pretreating a blood vessel, prior to grafting, to create a BMVG to improve vein graft survival. In some instances, the methods herein comprise irrigating a blood vessel to be grafted with a buffer; perfusing the buffer into the blood vessel without increasing intravasal pressure sufficient to distend the blood vessel; and instilling a solution of oligo-L-arginine through the lumen of the blood vessel without distending the blood vessel. The methods may also comprise contacting an exterior surface of the blood vessel with the solution of oligo-L-arginine. The buffer can comprise an organic acid or a salt thereof. The solution can also comprise an organic acid or a salt.

The method can further comprise contacting the blood vessel with the solution for a period of time. The period of time can be at least 5 minutes and up to 60, 55, 50, 45, 40, 35, 30, 25, 20, 15, or 10 minutes. 10 minutes is the preferred incubation time.

Following incubation and before implantation of the BMVG, the vein to be grafted may be flushed with the same solution that was used to apply the oligo-L-arginine but lacking oligo-L-arginine. Following flushing, the BMVG may be placed in the same solution absent the oligo-L-arginine until used in the vein graft bypass surgery. The surgical interposition grafting preferably takes place in accordance with standard operating proceedings.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

The present disclosure contemplates compositions and methods for reducing the incidence of vein graft disease and/or processes leading to vein graft disease such as thrombosis, intimal hyperplasia, and atherosclerosis. Such compositions include solutions that can be applied ex vivo to a vein graft (VG) prior to surgery or delivery of the VG to the subject, thereby resulting in a biologically modified vein graft (BMVG) with reduced incidences of occlusion or damage to the vein graft after surgery.

Graft diseases, such as saphenous vein graft disease, can cause acute and chronic occlusion that can lead to failure of the graft. Saphenous vein graft disease generally comprises three distinct temporal phases: thrombosis, intimal hyperplasia, and atherosclerosis.

After the bypass surgery, thrombosis can be the principal underlying mechanism for early stage graft occlusion. The thrombosis can be caused by a combination of alterations in the vessel wall, changes in blood rheology, and altered flow dynamics. After the initial stage, intimal hyperplasia, defined as the accumulation of smooth muscle cells and extracellular matrix in the intimal compartment, can be a major disease process in the graft. Intimal hyperplasia can be induced by proliferation and migration of smooth muscle cells into the intima, leading to a progressive increase in intimal fibrosis and a reduction in cellularity. In the later stage, the dominant process underlying the attrition of saphenous vein grafts and subsequent failure may be atherosclerosis, which can be initiated by luminal loss due to intimal hyperplasia.

Nitric oxide (NO) may play important roles in the inhibition of the three processes as it has anti-thrombotic properties from inhibition of platelet activation and anti-proliferative effects against smooth muscle cells. Hence, upregulation of NO production may present a therapeutic opportunity for vascular graft treatment to prevent vein graft disease. Because L-arginine is the limiting substrate in the enzymatic production of NO, graft survival may be improved by providing a pool of intracellular L-arginine. This pool of intracellular L-arginine can serve as the substrate for nitric oxide synthase (NOS) to produce nitric oxide (NO). Because oligo-L-arginines have the ability to cross cell membranes, they may be utilized for vascular graft pretreatment prior to implantation to provide this pool of intracellular L-arginine.

I. Vascular Grafts

The biologically modified vascular grafts contemplated herein can be used in any type of vascular bypass surgery or in other types of surgeries. A vascular graft surgery can be a cardiac arterial bypass graft, a peripheral arterial bypass graft (PABG), a lower extremity arterial bypass graft, an aortic bypass graft, a cerebral artery bypass graft, an aorto-iliac arterial bypass graft, an aorto-femoral arterial bypass graft, a fem-fem arterial bypass graft, an aorto-mesenteric arterial, or an ax-fem arterial bypass graft. The cardiac bypass can be a coronary arterial bypass graft (CABG). The PABG can be done on the aorta, an artery in the hip, an artery behind the knee, an artery in lower leg, or an artery in the armpit among other blood vessels. The vascular graft may be a composite graft. The vascular graft can be sequentially grafted to multiple targets. The vascular graft may be a human vascular graft.

The harvested graft can be a vein. The vein can be a saphenous vein. The vein can be the great saphenous vein. The vein can be the lesser saphenous vein. A saphenous vein can be harvested from the lower leg. The harvested graft can also be an artery. The artery can be a thoracic artery. The artery can be a mammary artery. The artery can be a radial artery in the arm. In some cases, the harvested graft is harvested from a human.

II. Compositions

Disclosed herein are compositions, and methods of using such compositions, for improving vascular graft survival. The compositions herein may be in a dry form that can be reconstituted into a liquid form. Alternatively, the compositions herein can be in a liquid form such as a solution.

(a) Arginine

The compositions herein comprise one or more L-arginines. The L-arginines can be part of an oligomer, such as an oligo-L-arginine. An oligo-L-arginine may have 6-15 L-arginine residues. Such L-arginine residues can be consecutive or non-consecutive. As such, the oligo-L-arginine can be a homopolymer, heteropolymer, or a copolymer with L-arginine as the major component. Preferably, the oligo-L-arginine is a nona-L-arginine.

Other embodiments include an oligo-L-arginine that comprises at least 5, 6, 7, 8, 9, 10, 11, 12, 13, 14, or 15 L-arginine residues. The oligo-L-arginine can comprise at least 15, 20, 25, or 30 residues in total (both L-arginine and non-arginine residues). Preferably, the oligo-L-arginine comprises more than five L-arginine residues. Preferably, the oligo-L-arginine comprises less than sixteen L-arginine residues. The oligo-L-arginine can comprise six L-arginine residues or seven L-arginine residues. The oligo-L-arginine may comprise nine L-arginine residues. The oligo-L-arginine can comprise up to fifteen L-arginine residues. The oligo-L-arginine herein can have the L-arginine monomers be consecutive residues. The oligo-L-arginine can comprise L-arginine residues that are non-consecutive residues.

In some instances, the oligo-L-arginine comprises nine L-arginine residues with the following structure:

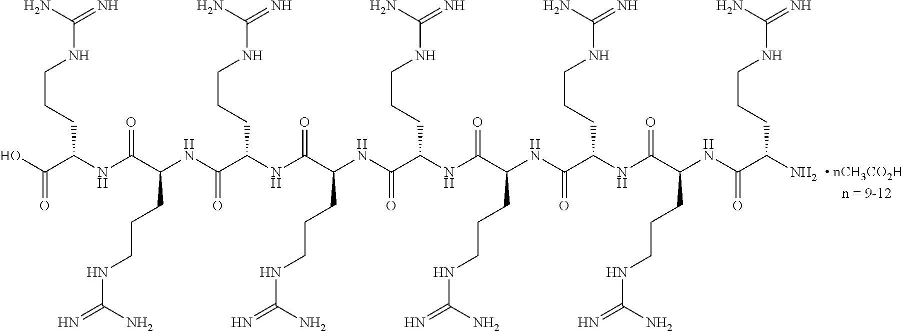 [SMILES: N=C(N)NCCC[C@H](NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@@H](N)CCCNC(=N)N)C(=O)O]

Contemplated herein are also salt forms of any of the oligo-L-arginines described herein.

The concentration of the active agents can be one that is sufficient to create a Biologically Modified Vein Graft that is resistant to at least one of thrombosis, intimal hyperplasia, and atherosclerosis of the vascular graft after surgery. In some instances, a composition comprises an oligo-L-arginine, such as a nona-L-arginine, at a concentration between 10 μM and 200 μM, or between 50 μM and 150 μM. In some instances, a preferred composition comprises an oligo-L-arginine, such as a nona-L-arginine, at a concentration that is about 100 μM. The term “about” shall mean+/−10%, 9%, 8%, 7%, 6%, 5%, 4%, 3%, 2%, or 1%. Alternatively, a solution herein can have a concentration of the oligo-L-arginine of about 10 μM, or more than 10 μM. Moreover, a solution herein can have a concentration of the oligo-L-arginine that is less than 250 μM.

(b) Buffers/Solutions

Any of the solutions herein can be in a buffer. For example, a solution herein can be in Lactated Ringer's solution, a GALA™/DuraGraft™ type Preservation Solution lacking heparin, a Perfadex™ perfusion solution, TiProtect™, acetate buffer, BES-buffered saline, bicine, CAPS, carbonate-bicarbonate buffer, CHES, citrate buffer, citrate buffer, diethanolamine, EBSS (magnesium, calcium, phenol red), glycine-HCl buffer, glycine-sodium hydroxide buffer, HBSS (Hank's Balanced Salt Solution), HEPES buffer, HEPPSO, HHBS (Hank's Buffer with HEPES), hydrochloric acid-potassium chloride buffer, imidazole-HCl buffer, maleic acid, MES, MOPS buffer, sodium borate buffer, TAE buffer, TAE, TBS, TE buffer, tricine, Tris buffer, or Trizma buffer. The solution can comprise plasma or plasma derivative. In any of the embodiments herein, the buffer is not phosphate buffered saline (PBS).

In some instances, a solution herein comprises an oligo-L-arginine, such as a nona-L-arginine, and one or more organic molecules such as an organic acid or salt thereof. An organic molecule contemplated herein can be a C2-C20 molecule, or preferably a C3-C10 molecule. In one example, the organic molecule is a lactate. The lactate can be sodium lactate. The lactate may be a racemic mixture of D(−)- and L(+)-isomers. In some cases, the lactate is the L(+)-isomer, herein “L-lactate”. Such lactate can be part of a solution such as a Lactated Ringer's solution. Thus, contemplated herein is a Lactated Ringer's solution comprising an oligo-L-arginine. The oligo-L-arginine is preferably a nona-L-arginine. The concentration of the oligo-L-arginine in the solution is about 100 μM. The pH of the solution is 6.5.

Other organic molecules, acids or salts thereof contemplated herein include, but not limited to: formic acid, glyoxilic acid, oxalic acid, acetic acid, glocolic acid, acrylic acid, pyruvic acid, malonic acid, propanoic acid, hydroxypropanoic acid, lactic acid, glyceric acid, fumaric acid, maleic acid, oxaloacetic acid, crotonoic acid, acetoacetic acid, 2-oxobutanoic acid, methylmalonic acid, succinic acid, malic acid, L-tartaric acid, DL-tartaric acid, meso-tartaric acid, dihydroxytartaric acid, butanoic acid, isobutanoic acid, hydroxybutanoic acid, itaconic acid, mesaconic acid, oxoglutaric acid, glutaric acid, methylsuccinic acid, valeric acid, isovaleric acid, pivalic acid, phenol, cis-aconitic acid, trans-aconitic acid, ascorbic acid, L-ascorbic acid, citric acid, isocitric acid, adipic acid, caproic acid, benzoic acid, salicylic acid, gentisic acid, protocatechuic acid, gallic acid, cyclohexanecarboxylic, pimelic acid, phthalic acid, isophthalic acid, terephthalic acid, phenylacetic acid, toluic acid, m-toluic acid, p-toluic acid, mandelic acid, homogentistic acid, suberic acid, octanoic acid, cinnamic acid, and nonanoic acid. The salt can be diammonium hydrogen citrate, triammonium citrate, calcium acetate, calcium formate, calcium hydrogen citrate, calcium lactate, iron(II) formate, dipotassium hydrogen citrate, tripotassium citrate, potassium acetate, potassium formate, potassium dihydrogen citrate, potassium lactate, magnesium acetate, magnesium formate, magnesium hydrogen citrate, magnesium lactate, disodium hydrogen citrate, trisodium citrate, sodium acetate, sodium formate, sodium dihydrogen citrate, sodium ascorbate, sodium lactate, ammonium acetate, ammonium formate, ammonium dihydrogen citrate, ammonium ascorbate, or ammonium lactate. A preferred formulation comprises a solution with an oligo-L-arginine and sodium lactate.

In some embodiments, the solution comprises one or more oligo-L-arginine or salts thereof, an organic acid or a salt thereof, and at least one of a lactate ion, a sodium ion, a potassium ion, a calcium ion, and a chloride ion. The compositions can contain one or more of sodium lactate, sodium chloride, potassium chloride, and calcium chloride. In other cases, the solution may be devoid of phosphate ions. The solution may be devoid of disodium phosphate and/or monopotassium phosphate. The pH of the solution can be between 6.0 to 7.5. The pH of the solution is preferably 6.5.

The solution can further comprise one or more other agents such as, e.g., MgCl2, KH2PO4, MgSO4, NaHCO2, Na2HPO4, D-glucose, glutathione, L-ascorbic acid, trometamol, CaCl2, dextran 40, and NaNO3, alpha-ketoglutarate, aspartate, N-acetyly-histidine, glycine, alanine, tryptophan, sucrose, glucose, deferoxamine, and N-Hydroxy-3,4-dimethoxy-N-methylbenzamide.

In some cases, the solution does not contain an antibiotic. In some cases, the solution does not contain heparin. In some cases, the solution does not contain and antibiotic and does not contain heparin.

Disclosed herein are kits comprising a dry formulation comprising oligo-L-arginine and one or more of the buffers and/or agents disclosed herein. For example, a kit can comprise a first container containing a dry formulation comprising an oligo-L-arginine, such as a nona-L-arginine. The kit can further comprise a second container containing a solvent and an instruction comprising a written direction giving one or more steps on combining the dry formulation and the solvent to form a final solution. The solvent may comprise an organic molecule such as organic acid or a salt thereof. In some instances, the solvent is a Lactated Ringer's Solution. The solvent may be a solvent other than PBS. In some cases, a kit may comprise a dry formulation comprising oligo-L-arginine, an organic molecule such as an organic acid or a salt thereof, and/or one or more calcium ions, and an instruction comprising a written direction giving one or more steps on combining the dry formulation with a solvent to form a final solution. The solvent may be an aqueous solvent. In some cases, the solvent may be water. Combining the oligo-L-arginine and the solvent results in a final solution with a concentration of oligo-L-arginine that is between 10-200 or 50-150 or at about 100 μM.

III. Ex Vivo Protocol (Methods of Use)

Treatment with the composition can be sufficient to result in an increase in nitric oxide production. The concentration of the active agents can be one that is sufficient to result in improved vascular graft survival. Treating a vessel with the composition herein can be sufficient to inhibit occlusion due to vein graft disease of the vascular graft after grafting. The treatment with the composition can be sufficient to keep the vascular graft patent, or not occluded, for at least 1 month, 2 months, 3 months, 6 months, 1 year, 2 years, or 3 years after grafting. Treatment with the solution can be sufficient to keep the vascular graft patent for at least about 5 years, 10 years, or 20 years after grafting.

The methods disclosed herein can be implemented for ex vivo treatment of a vascular graft prior to grafting to create a BMVG. The pre-grafting methods of improving vascular graft survival can comprise: irrigating a blood vessel to be grafted with a buffer; perfusing the buffer into the blood vessel to reach an intravasal pressure that does not distend the blood vessel; and instilling a solution comprising one or more oligo-L-arginine(s) through a lumen of the blood vessel without distending the blood vessel. The method can further comprise contacting the blood vessel with the solution for a period of time after which time and before implantation, the BMVG may be flushed to remove residual oligo-L-arginine solution before implantation.

The buffer used for irrigation and perfusion can comprise an organic acid or a salt thereof. The organic acid or salt can be those described in the compositions. The solution instilled into the lumen is preferably one or more of the compositions described herein. For example, a lumen of the blood vessel can be perfused with a solution comprising oligo-L-arginine(s) and a salt of an organic acid.

The buffer is also used to perfuse the vessel to check for leaks. The leaks may be repaired if a surgeon determines that the vessel is capable of being used in a bypass surgery after the repairs. If the vessel is not capable of being used for bypass surgery even after the repairs, the vessel may be discarded, and another blood vessel may be identified and isolated. Subsequently, once the vein graft is identified as capable of being used as a bypass, the vein graft is immersed in a solution comprising oligo-L-arginine(s). The solution can also comprise an organic acid or a salt thereof. Following incubation of the vein graft in the solution containing the oligo-L-arginine and before implantation, the BMVG is flushed to remove residual oligo-L-arginine solution.

The blood vessel can be perfused without using a pressure monitoring device. Alternatively, the blood vessel can be perfused with the use of a pressure monitoring device. The intravasal pressure of the perfusion is not high enough to cause distension of the vessel. In some cases, the intravasal pressure may be maintained below 30 mmHg. The intravasal pressure may be maintained below 10 mmHg or 5 mmHg.

The length of ex vivo treatment can vary to improve vascular graft survival. Disclosed herein is a method of improving vascular graft survival comprising contacting a blood vessel to be grafted, ex vivo, to any of the compositions herein (e.g., a solution comprising oligo-L-arginine and an organic acid) for up to 45 minutes. In some instances, the contacting occurs for up to 30 minutes. In some instances, the contacting occurs for up to 15 minutes. In the preferred embodiment, the contacting occurs for 10 minutes. Following this contacting, the BMVG may be flushed to remove residual oligo-L-arginine solution.

Increased nitric oxide production can be an indication of improved vascular graft survival. Disclosed herein is a method of increasing nitric oxide production in a vascular graft ex vivo comprising contacting the vascular graft, prior to grafting, with a solution comprising oligomers of L-arginine and an organic acid or a salt thereof.

IV. Treatment Subjects

The method of improving vascular graft survival can be conducted on pigs, dogs, rabbits, rats, mice, or other animals. The method can be conducted in any mammal. The mammal can be a human. The mammal can be a primate. The primate may be a chimpanzee or a macaque.

The method of improving vascular graft survival can be conducted on human patients undergoing vascular bypass surgery. A vascular graft surgery can be a CABG, a peripheral arterial bypass graft (PABG), a lower extremity arterial bypass graft, an aortic bypass graft, a cerebral artery bypass graft, an aorto-iliac bypass graft, an aorto-femoral bypass graft, a fern-fern bypass graft, an aorto-mesenteric, or an ax-fem bypass graft. The PABG can be done on the aorta, an artery in the hip, an artery behind the knee, an artery in a lower leg, and an artery in an armpit among other blood vessels. The vascular graft can be a composite graft. The vascular graft can be a sequential graft to multiple targets.

In some cases, the treatment subject may be a patient with multi-vessel Coronary Artery Disease (CAD). In some cases, the treatment subject may be a patient with severe triple coronary artery disease In some cases, the treatment subject may be a patient with greater that 50% left main, severe triple coronary artery disease. In some cases, the treatment subject may be a patient with single or double vessel disease. In some cases, the treatment subject may be a patient deemed not amendable to angioplasty and/or stent. In some cases, the treatment subject may be a patient who has had previously placed stents with stent failure. In some cases, the treatment subject is a patient who is considered a candidate for saphenous vein harvest for utilization as a bypass graft.

In some cases, the treatment subject may be administered a statin following the graft procedure. In some cases, the statin may be administered for about 1 month, about 2 months, about 3 months, about 4 months, about 5 months, about 6 months, about 12 months, about 18 months, about 24 months, about 30 months, about 36 months, about 4 years, about 5 years or more than 5 years after the graft procedure. Examples of statins may include, but are not limited to, atorvastatin, fluvastatin, lovastatin, pitavastatin, pravastatin, rosuvastatin, and simvastatin. In some cases, the treatment subject may be administered simvastatin.

In some cases, the treatment subject may be administered an antiplatelet agent following the graft procedure. In some cases, the antiplatelet therapy may be administered for about 1 month, about 2 months, about 3 months, about 4 months, about 5 months, about 6 months, about 12 months, about 18 months, about 24 months, about 30 months, about 36 months, about 4 years, about 5 years or more than 5 years after the graft procedure. The antiplatelet agent may be an irreversible cyclooxygenase inhibitor such as aspirin or triflusal (Disgren). The antiplatelet agent may be an adenosine diphosphate (ADP) receptor inhibitor such as cangrelor (Kengreal), clopidogrel (Plavix), prasugrel (Effient), ticagrelor (Brilinta), or ticlopidine (Ticlid). The antiplatelet agent may be a phosphodiesterase inhibitor, such as cilostazol (Pletaal), protease-activated receptor-1 (PAR-1) antagonists, or vorapaxar (Zontivity). The antiplatelet agent may be a glycoprotein IIB/IIIA inhibitor such as abciximab (ReoPro), eptifibatide (Integrilin), or tirofiban (Aggrastat). The antiplatelet agent may be an adenosine reuptake inhibitor such as dipyridamole (Persantine). The antiplatelet agent may be a thromboxane inhibitor, such as a thromboxane synthase inhibitor or a thromboxane receptor antagonist such as terutroban. In some cases, the antiplatelet agent is aspirin.

V. End Points

The compositions herein can be used for improved vascular graft survival. This can be achieved by inhibition of at least one of thrombosis, intimal hyperplasia, and atherosclerosis. The inhibition of intimal hyperplasia can be determined by the reduction in a ratio of intima-media thicknesses. The inhibition of intimal hyperplasia can also be determined by the reduction in the intima-media area like cross-sectional surface area. The reduction in the ratio of intima-media thicknesses and luminal narrowing can be determined by using tools such as computed tomographic angiography.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means without the exercise of inventive capacity and without departing from the scope of the invention.

### Example 1: Ex Vivo Vascular Graft Pretreatment

A surgeon performing coronary artery bypass grafting on a patient removes a saphenous vein from the lower leg of the patient. The surgeon places a vessel cannula in the vein and irrigates the vein with Lactated Ringer's Solution (LRS). The proximal saphenous vein is then suture ligated and placed in an LRS basin for preparation. The surgeon then ligates and clips all side branches of the saphenous vein and perfuses the saphenous vein with LRS. If necessary, a 7-0 prolene suture is utilized to repair leaks. Afterwards, using a syringe, the surgeon instills nona-L-arginine in LRS solution through the lumen of the saphenous vein without distention. The saphenous vein is placed in a basin of nona-L-arginine solution and treated for 10 minutes. The treated saphenous vein is then flushed with LRS without distension, using a syringe, and placed in LRS solution until implantation.

### Example 2: Results of a Phase 1/2 CABG Study

57 of the initial 80 subjects enrolled in the safety cohort of the double-blinded Phase 1/2 clinical trial were available to be evaluated at one year. There were no drug-related Serious Adverse Events in the safety cohort. Subjects were evaluated at one month and again at one year by Computed Tomographic Angiography (CTA). Vein grafts in 12 of the 30 subjects in the vein graft (VG) control group had occlusion (10) or damage to the VG (2), whereas only 7 of 27 subjects in the Biologically Modified Vein Graft (BMVG) group had occlusion (5) or damage (2) to a BMVG (see Table 1 below). Of the 53 VGs and the 46 BMVGs evaluated at 1 year, there were 10 (19%) and 6 (13%) failures, respectively.

The BVMGs were created by excising and then incubating the patient's saphenous (lower leg) vein in a 100 μM solution of nona-L-arginine in Lactated Ringer's Solution (LRS) for 10 minutes followed by flushing with LRS prior to implantation. The nona-L-arginine rapidly translocated into the endothelium and served as a substrate for the enzyme nitric oxide synthase (NOS) to allow nitric oxide (NO) production.

The presence of NO allows muscle cells to form around the vein graft and keeps the vein graft open and dilated. In the absence of L-arginine there is an injury response due to the arterial pressure, amongst other insults to the vein graft, that generates reactive oxygen species that causes thrombosis and intimal hyperplasia within the vein graft that leads to re-occlusion of the vein graft due to vein graft disease.

TABLE 1
Trial Outcomes
Per-Patient Analysis—1 Year

| | | | Failed | | Failed or Degraded | |
|---|---|---|---|---|---|---|
| Group | # Subjects | Δ Days | # | % | # | % |
| VG | 30 | 381 | 10 | 33.3% | 12 | 40.00% |
| BMVG | 27 | 381 | 5 | 18.5% | 7 | 25.93% |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of improving vascular graft survival comprising contacting a blood vessel to be grafted, prior to grafting, ex vivo with a solution comprising one or more oligo-L-arginine, or salts thereof, and an organic acid or a salt thereof.

2. The method of claim 1, wherein the organic acid or the salt thereof is a C3-C10 organic acid or a salt thereof.

3. The method of claim 2, wherein the C3-C10 organic acid is lactic acid or a salt thereof.

4. The method of claim 3, wherein the C3-C10 organic acid is L-lactic acid or a salt thereof.

5. The method of claim 1, wherein the solution comprises at least one of a lactate ion, a sodium ion, a potassium ion, a calcium ion, a chloride ion, MgCl2, KH2PO4, MgSO4, NaHCO2, Na2HPO4, D-glucose, glutathione, L-ascorbic acid, trometamol, CaCl2, dextran 40, and NaNO3, alpha-ketoglutarate, aspartate, N-acetyly-histidine, glycine, alanine, tryptophan, sucrose, glucose, deferoxamine, and N-Hydroxy-3,4-dimethoxy-N-methylbenzamide.

6. The method of claim 5, wherein the solution consists essentially of sodium lactate, sodium chloride, potassium chloride, and calcium chloride.

7. The method of claim 1, wherein the solution does not comprise at least one of a phosphate ion, a disodium phosphate, a monopotassium phosphate, a heparin, and an antibiotic.

8.-13. (canceled)

14. The methods of claim 1, wherein the one or more oligo-L-arginine is a nona-L-arginine.

15.-45. (canceled)

46. A method of keeping a vascular graft patent in a subject for at least 1 month after a graft surgery comprising contacting a blood vessel to be grafted, prior to grafting, ex vivo with a solution comprising one or more oligo-L-arginines or salts thereof.

47. The method of claim 46, wherein the vascular graft is patent for at least 2 months.

48.-61. (canceled)

62. The method of claim 46, wherein the oligo-L-arginine is a nona-L-arginine.

63.-85. (canceled)

86. A solution for improving vascular graft survival comprising:
an oligo-L-arginine or salt thereof, and
an organic acid or a salt thereof.

87. The solution of claim 86, wherein the organic acid or the salt thereof is a C3-C10 organic acid or the salt thereof.

88. The solution of claim 87, wherein the C3-C10 organic acid or the salt thereof is lactic acid or a salt thereof.

89.-90. (canceled)

91. The solution of claim 86, wherein the solution does not comprise at least one of a phosphate ion, a disodium phosphate, and a monopotassium phosphate.

92.-95. (canceled)

96. The solution of claim 95, wherein the oligo-L-arginine is a nona-L-arginine.

97. The solution of claim 86, wherein the solution is a Lactated Ringer's solution.

98.-115. (canceled)

116. The method of claim 1, wherein the blood vessel to be grafted is a human blood vessel.

117. The method of claim 46, wherein the subject is a human subject.

118. The method of claim 68, wherein the blood vessel to be used as a vascular graft is a human blood vessel.

119. (canceled)
